(19) United States
(12) Patent Application Publication
(10) Pub. No.: US 2024/0418736 A1
(43) Pub. Date: Dec. 19, 2024

(54) Assay Sample Preparation for Reagent Uniformity, Particularly Blood

(71) Applicant: Essenlix Corporation, Monmouth Junction, NJ (US)

(72) Inventors: Stephen Y. CHOU, Princeton, NJ (US); Wei DING, Princeton, NJ (US)

(73) Assignee: Essenlix Corporation, Monmouth Junction, NJ (US)

(21) Appl. No.: 18/697,622

(22) PCT Filed: Oct. 3, 2022
(86) PCT No.: PCT/US2022/045590

§ 371 Date: Apr. 1, 2024

Related U.S. Application Data
(60) Provisional application No. 63/251,470, filed on Oct. 1, 2021.

(51) Int. Cl. G01N 33/80; B01L 3/00; G01N 33/72

(52) U.S. Cl. CPC G01N 33/80; B01L 3/502715; G01N 33/721; B01L 2200/16; B01L 2300/0654; B01L 2300/0681; B01L 2400/0481

(57) ABSTRACT

The disclosure provides a kit and method using the kit for analyzing a sample, particularly blood. The kit may comprise a device (a) for sample collecting, mixing, staining, and transferring and a device (b) for sample distribution and imaging device. The device (a) comprises part (1) and an optional part (2) connected to each other. The part (1) is a capillary tube with reagent coated inside. The part (2) is used for providing an external force to suck in and/or push out the sample. The device (b) comprises two plates opposing to each other and defining an interior cavity, and a sample inlet. The interior cavity is in fluid communication with the exterior liquid sample contact area. The liquid sample flows into the spacing between the two plates through the sample inlet and a sample landing Zone that is at the exterior location of the device (b).

### CROSS-REFERENCE TO RELATED APPLICATION

This application is a national entry of PCT/US22/45590, filed Oct. 3, 2022, which claims priority to the U.S. provisional application with Ser. No. 63,251/470, filed Oct. 1, 2021, the entire contents of which are incorporated herein by reference.

### FIELD

The disclosure relates to a device and a kit of preparing and analyzing a sample, particular blood, and the methods for using the same.

### BACKGROUND

In many bio/chemical assays, it involves mixing a reagent with a sample. Often the assay requires a uniform mixing for a desirable results. Therefore, there is a need to have a method and a device that can provide easy, fast and uniform mixing of a reagent with a sample.

### SUMMARY

The present invention provides, among other things, devices and methods for mixing of a reagent with a sample and for perform assaying (including imaging and analysis of the sample). In an embodiment, the kit includes: (i) a sample collecting and transferring device (device (a)), which is used for sample collection, sample mixing, sample staining and sample transferring, and (ii) a sample distribution and imaging device (device (b)). The device (a) comprises part (1) and an optional part (2) connected to each other, in which the part (1) is a capillary tube with an reagent coated inside, which is used for sample collection, mixture of the sample with pre-coated reagents inside the inner surface of the tube, and the part (2) is optional is used for providing an external force to suck in and/or push out the sample in the part (1). The device (b) includes two plates opposing to each other, in which the two plates define an interior cavity, and an exterior liquid sample contact area or sample inlet on an exterior location of the device (b). The interior cavity is in fluid communication with the exterior liquid sample contact area. The liquid sample flows into the spacing between the two plates from the exterior location through sample inlet and a sample landing Zone that is at the exterior location of the device (b) and connected to the sample inlet.

In an embodiment, a reagent pre-coated inside the cavity. In an embodiment, the reagent coated on the sample collecting and transferring device is surfactant as Zwittergent, anti-coagulation reagents as K₂EDTA. In an embodiment, a volume of the sample collecting and transferring device is 1 μl, 3 μl, 5 μl, 10 μl, 15 μl, 20 μl or in a range between any of these values. In an embodiment, the spacing height between the two plates is 2 μm, 3 μm, 5 μm, 8 μm, 10 μm or in a range between any of these values. In an embodiment, the reagent coated on the collecting and transferring device is surfactant as Zwittergent, anti-coagulation reagents as K₂EDTA. In an embodiment, the spacing height between two plates is 10 μm, 15 μm, 20 μm, 30 μm, 50 μm, 80 μm, 100 μm, 150 μm or in a range between any of these values. In an embodiment, the spacing height between two plates is 30 μm, 50 μm, 80 μm, 100 μm, 150 μm or in a range between any of these values. In an embodiment, the reagent coated on the collecting and transferring device is surfactant as Zwittergent, anti-coagulation reagent as K₂EDTA, fluorescence dye as Acridine orange, colorimetric dye as methylene blue. In an embodiment, the reagent coated on the collecting and transferring device is surfactant as Zwittergent, anti-coagulation reagent as K₂EDTA, fluorescence dye as Acridine orange and YOYO, colorimetric dye as methylene blue.

In an embodiment, the volume of the sample collection device is 1 μl, 3 μl, 5 μl, 10 μl, 15 μl, 20 μl or in a range between any of these values. In an embodiment, the spacing height between two plates is 1 μm, 5 μm, 8 μm, 10 μm, 15 μm, 20 μm, 30 μm, or in a range between any of these values. In an embodiment, there are several different spacing areas in the chamber.

In an embodiment, the difference between two spacing of each area is 0.5 μm, 1 μm, 2 μm, 3 μm, 5 μm, 10 μm, 20 μm, 30 μm, 50 μm, 100 μm, 150 μm, or in a range between any two of the values.

In an embodiment, the two plates are not aligned at their edges to create the flowing boundary of the sample by capillary force. In an embodiment, the sample collecting and transferring device and the sample distribution and imaging device each comprises an inner surface having a hydrophobic coating, hydrophilic coating, or both hydrophobic and hydrophilic coatings. In an embodiment, a coating is on at least an interior opposing surface of one or both of the plates. In an embodiment, the liquid sample for analyzing is pre-mixed with reagents before flowing into the device (a) or (b). In an embodiment, the reagent is coated with a slope distribution inside the device to balance the final concentration in the device (a) after adding the liquid sample.

In an embodiment, there is a slow releasing coating with the reagent inside the device (a), the slow releasing layer delaying the reagents dissolving into the liquid sample after the sample become static after flowing in. In an embodiment, the slowing releasing time is 3 second, 5 second, 10 second or in a range between any of these values. In an embodiment, the slow releasing materials comprise albpmin, carbomers, carboxymethyl cellulose, carrageenan, chitosan, dextrin, polyethylene glycol, polyvinylpyrrolidone, and/or polyvinyl alcohol.

In an embodiment, an acridine orange is coated onto one or both of the two plates. In an embodiment, a Zwittergent is coated onto one or both of the two plates. In an embodiment, a Methylene blue and a Zwittergent are coated onto one or both of the two plates. In an embodiment, an acridine orange and a Zwittergent are coated onto one or both of the two plates. In an embodiment, a YOYO dye and a Zwittergent are coated onto one or both of the plates.

In an embodiment, the device further comprises, on one or both plates, multi reagent layers including anti-conglutination, cell lysing, cell staining, release time control material layers, and their combinations.

In an embodiment, there is an array of spacers disposed between the two plates. In an embodiment, the spacers are attached on one of the plates or both plates, in which the spacers can be used as physical support of the two plates, the geometry reference, dimensional reference, and/or optical reference in the measurements.

In an embodiment, the lateral dimension of each spacer in each direction is the same or different. In an embodiment, the lateral dimension for each direction (x or y) is 1 nm or less, 3 nm or less, 5 nm or less, 7 nm or less, 10 nm or less, 20 nm or less, 30 nm or less, 40 nm or less, 50 nm or less, 100 nm or less, 200 nm or less, 500 nm or less, 800 nm or less, 1000 nm or less, 1 μm or less, 2 μm or less, 3 μm or less, 5 μm or less, 10 μm or less, 20 μm or less, 30 μm or less, 50 μm or less, 100 μm or less, 150 μm or less, 200 μm or less, 300 μm or less, or 500 μm or less, or in a range between any two of the values. In an embodiment, the period of spacer array is between 1 nm to 300 μm. In an embodiment, one dimension of each spacer is 0.1 mm, 0.2 mm, 0.5 mm, 1 mm, 2 mm, 5 mm, 10 mm, 20 mm or in a range between any of the two values. In an embodiment, the thickness variation of the spacers is less than 1%, less than 3%, less than 5%, less than 10% or in a range between any of the two values.

The disclosure also provide a method of performing biological and chemical assays, comprising the steps. In an embodiment, the method comprises
(1) providing the kit disclosed herein,
(2) collecting a sample into the sample collecting and transferring device, where the sample is mixed and stained by the reagent in the sample collecting and transferring device;
(3) transferring the sample from the sample collecting and transferring device by providing the external force to push out the sample to the sample landing Zone at the exterior location of the sample distribution and imaging device, where the sample flows into the cavity of the sample distribution and imaging device from the sample landing Zone and mix with reagent inside the sample distribution and imaging device, and distribute inside the sample distribution and imaging device; and
(4) imaging and analyzing the sample inside the sample distribution and imaging device.

In an embodiment, the liquid sample is whole blood, and an analyte to be analyzed is red blood cells, white blood cells and platelets. In an embodiment, wherein parameters measured in an imaging area is complete blood count, red blood cell count, hemoglobin, platelet count, white blood cell differentiation and count, and/or abnormal cell types.

### BRIEF DESCRIPTION OF THE DRAWINGS

A skilled artisan will understand that the drawings, described below, are for illustration purposes only. In some Figures, the drawings are in scale. For clarity purposes, some elements are enlarged when illustrated in the Figures. It should be noted that the Figures do not intend to show the elements in strict proportion. The dimensions of the elements should be delineated from the descriptions herein provided and incorporated by reference. The drawings are not intended to limit the scope of the present invention in any way.

FIG. 1 schematically illustrates an embodiment of device set and kit for analyzing a sample, particularly blood in the cross-section view. The set contains two devices shown in FIGS. 1a and 1b. FIG. 1 a schematically illustrates a sample collecting and transferring device, which is used for sample collection, sample mixing, sample staining and sample transferring. The device shown in FIG. 1a has two parts connected to each other. Part (1) is a capillary tube with reagent coated inside, which is used for sample collection by from the inlet, mixture of the sample with pre-coated reagents inside the inner surface of the tube. Part (2) is optional, which can be a gas pμmp, a pipette or a plunger, which is used for providing the external force to suck in and/or push out the sample in the Part (1). FIG. 1b schematically illustrates an embodiment of a sample distribution and imaging device. An embodiment of the sample distribution and imaging device may comprise: two plates oppose to each other, wherein the opposed plates define an interior cavity; an exterior liquid sample contact area or sample inlet on an exterior location of the device, wherein the interior cavity is in fluid communication with the exterior liquid sample contact area; the liquid sample can flow into the spacing between the two plates from the exterior location through sample inlet. There is a sample landing Zone at the exterior location of the device which connected to the sample inlet. Optionally, there are reagents pre-coated inside the cavity. In usage of the device set, the sample are first collected into the device of FIG. 1a, then transferred to the landing Zone at the device of FIG. 1b, and finally flow into the cavity in the device of FIG. 1b for imaging and analyzing.

FIG. 2 schematically illustrates a flow chart of using the device set and kit for analyzing a sample, particularly whole blood, in accordance with an embodiment.

FIG. 3 illustrates a set of devices for analyzing a sample, particularly blood, in accordance with an embodiment. (a) Photo of an example sample collecting and transferring device, which is used for sample collection, sample mixing, sample staining and sample transferring. (b) Photo and structure of a sample distribution and imaging device. The device comprising: two plates oppose and are bonded to each other to form the spacing, the whole blood sample can flow into the spacing between the two plates from the exterior location through sample inlet. (c) The bright field image example of RBC and HgB inside the device. (d) The fluorescence image example of stained whole blood film inside the device.

FIG. 4 illustrates a set of devices for collection, mixing and analyzing a sample, particularly blood, in according with an embodiment. (a) Schematic of a collection tube with tube to suck in sample and plunger to push out the sample. (b) A mix enhancer tube, which can be installed on the outlet of collection tube and has a mixing chamber which can mix the sample with reagent inside when passing through. (c) Schematic of assembled collection tube with mix enhancer tube.

### DETAILED DESCRIPTION

The following detailed description illustrates certain embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention. In using a lateral flow microfluidic channel sample holder, one of the issues is related to a mixing of reagent into the sample. It is desirable for a simple device and method to make the mixing uniform. The prior methods with reagent coated in the lateral fluidic channel often lead to a nonuniform mixing, because the portion of the sample that flows in the sample holder first may have reagent than the portion of the sample flows in later.
A-1 According to the present invention, a method of preparing and analyzing a whole blood with rapid simple sample preparation steps, without dilution, and with improved reagent mixing uniformity, improved cell distribution in blood, and reduced cell aggregation, comprising: obtaining a sample collector;
   obtaining a sample analyzing card;
   pricking skin of a subject to cause a bleeding (e.g., prick a finger);
   collecting the blood from the skin using the sample collector;
   depositing, after collecting the whole blood sample, without diluting the sample, without shaking the sample, within in 3 (three) min after the sample collection, the blood sample at the inlet of a lateral microfluidic channel the analyzing card; wherein the analyzing card sucks at least a part of the sample inside of the card; and
   analyzing the at least part of the blood sample inside of the card;
   wherein the sample collector comprising:
      (i) a capillary tube that has a reagent inside the tube and suck a liquid sample from outside the tube into the inside of the tube using capillary force;
      (ii) a plunger that can push the sample sucked inside the capillary tube outside of the tube; and
      (iii) the reagent comprises zwittergent of a concentration selected in the range of 0.2 mg/mL to 10 mg/mL, acridine orange of a concentration selected in the range of 0.2 mg/mL to 3 mg/mL, or any combination for improving the cell distribution in the blood with less aggregation and for staining the cell; and
   wherein the analyzing card comprises two plates forming the lateral microfluidic channel that has a channel height of 200 μm or less.
A-2 According to the present invention, a kit of preparing and analyzing a whole blood with rapid simple sample preparation steps, without dilution, and with improved reagent mixing uniformity, improved cell distribution in blood, and reduced cell aggregation, comprising:
   a sample collector; and
   a sample analyzing card;
   wherein the sample collector comprising:
      (i) a capillary tube that has a reagent inside the tube and suck a liquid sample from outside the tube into the inside of the tube using capillary force;
      (ii) a plunger that can push the sample sucked inside the capillary tube outside of the tube; and
      (iii) the reagent comprises zwittergent of a concentration selected in the range of 0.2 mg/mL to 10 mg/mL, acridine orange of a concentration selected in the range of 0.2 mg/mL to 3 mg/mL, or any combination for improving the cell distribution in the blood with less aggregation and for staining the cell; and
   wherein the analyzing card comprises two plates forming the lateral microfluidic channel that has a channel height of 200 μm or less.

In some embodiments, the sample analyzing card further comprising a reagent on one or both of the two plates . . . .

In some embodiments, the channel height is 30 μm.

In some embodiments, the channel height is 10 μm.

In some embodiments, the channel height is 5 μm.

In some embodiments, the card has multiple of lateral flow microfluidic channels on sample analyzing card.

In some embodiments, the card has multiple of lateral flow microfluidic channels on sample analyzing card, wherein the channel of different channel height of 5 μm and 30 μm.

In some embodiments, the card has multiple of lateral flow microfluidic channels on sample analyzing card, wherein at least a first channel has channel height in range of 3 μm to 10 μm, and at least a second channel has a channel height in range of 20 μm to 40 μm.

### Range-1

In some embodiment, a concentration of zwittergent is in the range of 0.2 mg/mL to 10 mg/mL, the concentration of acridine orange is in the range of 0.2 mg/mL to 3 mg/m, or any combinations.

### Range-2

In some embodiment, a concentration of zwittergent is in the range of 0.5 mg/mL to 6 mg/mL, the concentration of acridine orange is in the range of 0.5 mg/mL to 2 mg/m, or any combinations.

### Range-3

In some embodiment, a concentration of zwittergent is in the range of 1 mg/mL to 5 mg/mL, the concentration of acridine orange is in the range of 0.6 mg/mL to 1.5 mg/m, or any combinations.

### Range-4

In some embodiment, a concentration of zwittergent is in the range of 1 mg/mL to 2 mg/mL, the concentration of acridine orange is in the range of 0.6 mg/mL to 1.5 mg/m, or any combinations.

### Range-5

In some embodiment, a concentration of zwittergent is in the range of 4 mg/mL to 6 mg/mL, the concentration of acridine orange is in the range of 0.6 mg/mL to 1.5 mg/m, or any combinations.

In some embodiments, the sample is deposited from the collection tube on the sample analyzing card within in 2 (two) min after the sample collection,

In some embodiments, the sample is deposited from the collection tube on the sample analyzing card within in 1 (one) min after the sample collection.

According to the present invention, a kit for making a sample that flow inside of a lateral flow microfluidic channel with a channel height of 100 micron or less and a width of 200 μm or wider, comprising: a lateral flow microfluidic channel and a sample collection tube

Wherein the lateral flow microfluidic channel comprising:
(i) a channel that has a height of 100 micron or less and a width of 200 μm or wider;
(ii) an sample inlet of the channel,
(iii) a sample landing area next to the inlet

wherein the sample collection tube comprising:
(iv) a capillary tube that has reagent inside of the tube and can suck a liquid sample outside of the tube into the tube using capillary force;
(v) a plunger that can push the sample sucked inside the capillary tube outside of the tube;

wherein the amount of the reagent inside the tube is configured to
A-2 According to the present invention, a method of mixing

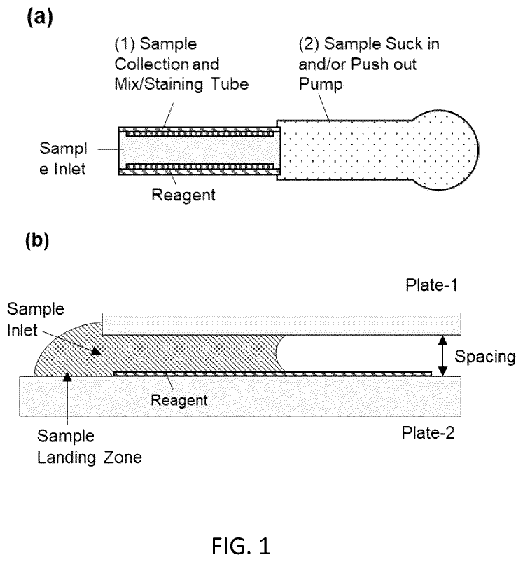

In one or more embodiment, the present invention provides, for example (FIG. 1):

One embodiment of device set and kit for analyzing a sample, particularly blood. The set contains two devices:
(a) A sample collecting and transferring device, which is used for sample collection, sample mixing, sample staining and sample transferring. The device has two parts connected to each other. Part (1) is a capillary tube with reagent coated inside, which is used for sample collection from the inlet, mixture of the sample with pre-coated reagents inside the inner surface of the tube. Part (2) is optional, which can be a gas pump, a pipette or a plunger, which is used for providing the external force to suck in and/or push out the sample in the Part (1).
(b) A sample distribution and imaging device. One embodiments of the device comprising: two plates oppose to each other, wherein the opposed plates define an interior cavity; an exterior liquid sample contact area or sample inlet on an exterior location of the device, wherein the interior cavity is in fluid communication with the exterior liquid sample contact area; the liquid sample can flow into the spacing between the two plates from the exterior location through sample inlet. There is a sample landing Zone at the exterior location of the device which connected to the sample inlet. Optionally, there are reagents pre-coated inside the cavity.

In usage of the device set, the sample are first collected into the device (a), then transferred to the landing Zone at device (b), and finally flow into the cavity in the device (b) for imaging and analyzing.

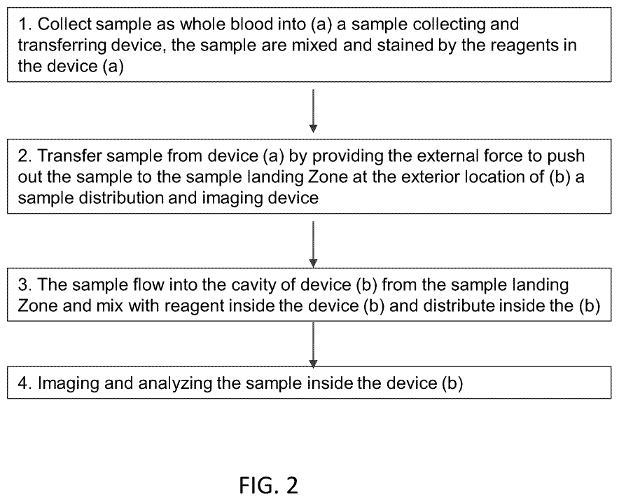

A method of performing biological and chemical assays using the device, comprising the steps of (FIG. 2):
(1) Collect sample as whole blood into (a) a sample collecting and transferring device, the sample are mixed and stained by the reagents in the device (a) (b) dropping the sample onto the exterior liquid sample contact area;
(2) Transfer sample from device (a) by providing the external force to push out the sample to the sample landing Zone at the exterior location of (b) a sample distribution and imaging device;
(3) The sample flow into the cavity of device (b) from the sample landing Zone and mix with reagent inside the device (b) and distribute inside the (b);
(4) Imaging and analyzing the sample inside the device (b).

### Sample, Analyzing Parameters and Corresponding Critical Ranges

In some embodiments, the liquid sample is whole blood, and the analyte to be analyzed is red blood cells, white blood cells and platelets.

In one embodiment, the parameters measured in imaging area is complete blood count including but not limit to white blood cell count, red blood cell count, hemoglobin, platelet count, white blood cell differentiation and count e.g., neutrophils, lymphocytes, monocytes, eosinophils and basophils—as well as abnormal cell types if they are present.

In some embodiment, for measuring red blood cell in the device:
the reagent coated on the sample collection device is surfactant as Zwittergent, anti-coagulation reagents as K₂EDTA;
the volume of the sample collection device is 1 μl, 3 μl, 5 μl, 10 μl, 15 μl, 20 μl or in a range between any of these values;
the spacing height between two plates is 2 μm, 3 μm, 5 μm, 8 μm, 10 μm or in a range between any of these values;
the preferred spacing height between two plates is 4 μm, 5 μm, 6 μm, or in a range between any of these values;
In some embodiment, for measuring hemoglobin in the device:
the reagent coated on the sample collection device is surfactant as Zwittergent, anti-coagulation reagents as K₂EDTA;
the volume of the sample collection device is 1 μl, 3 μl, 5 μl, 10 μl, 15 μl, 20 μl or in a range between any of these values;
the spacing height between two plates is 10 μm, 15 μm, 20 μm, 30 μm, 50 μm, 80 μm, 100 μm, 150 μm or in a range between any of these values;
the preferred spacing height between two plates is 30 μm, 50 μm, 80 μm, 100 μm, 150 μm or in a range between any of these values;
In some embodiment, for measuring white blood cell in the device:
the reagent coated on the sample collection device is surfactant as Zwittergent, anti-coagulation reagents as K₂EDTA, fluorescence dye as Acridine orange, colorimetric dye as methylene blue;
the volume of the sample collection device is 1 μl, 3 μl, 5 μl, 10 μl, 15 μl, 20 μl or in a range between any of these values;
the spacing height between two plates is 5 μm, 8 μm, 10 μm, 15 μm, 20 μm, 30 μm, 50 μm, 80 μm, 100 μm, 150 μm or in a range between any of these values;
the preferred spacing height between two plates is 10 μm, 15 μm, 20 μm, 30 μm, 50 μm, 80 μm, 100 μm, or in a range between any of these values; In some embodiment, for measuring platelets in the device:
the reagent coated on the sample collection device is surfactant as Zwittergent, anti-coagulation reagents as K₂EDTA, fluorescence dye as Acridine orange and YOYO, colorimetric dye as methylene blue;
the volume of the sample collection device is 1 μl, 3 μl, 5 μl, 10 μl, 15 μl, 20 μl or in a range between any of these values;
the spacing height between two plates is 1 μm, 5 μm, 8 μm, 10 μm, 15 μm, 20 μm, 30 μm, or in a range between any of these values;
the preferred spacing height between two plates is 1 μm, 2 μm, 5 μm, 10 μm, 15 μm, 20 μm, or in a range between any of these values;

In some embodiment, there are several different spacing areas in the chamber. The spacing heights can be combination of above.

The device of any prior device claim, wherein the spacing of each area is 1 μm, 2 μm, 3 μm, 5 μm, 10 μm, 20 μm, 30 μm, 50 μm, 100 μm, 150 μm or in a range between any two of the values.

The device of any prior device claim, wherein the preferred spacing of one area is 2 μm, 3 μm, 5 μm, 10 μm, or in a range between any two of the values.

The device of any prior device claim, wherein the preferred spacing of one area is 10 μm, 30 μm, 50 μm, 100 μm, or in a range between any two of the values.

The device of any prior device claim, wherein the difference between two spacing of each area is 0.5 μm, 1 μm, 2 μm, 3 μm, 5 μm, 10 μm, 20 μm, 30 μm, 50 μm, 100 μm, 150 μm, or in a range between any two of the values.

The device of any prior device claim, wherein the ratio of the manufacturing spacing height between two area is 1.1 fold, 1.2 fold, 1.5 fold, 2 fold, 3 fold, 5 fold, 10 fold, 30 fold, 50 fold, 100 fold, or in a range between any two of the values.

In one embodiment, the signal measured in imaging area is cell or particle numbers.

In one embodiment, the signal measured in imaging area is colorimetric intensity.

In one embodiment, the signal measured in imaging area is transmitted light intensity.

In one embodiment, the signal measured in imaging area is fluorescence signal intensity.

In one embodiment, the signal measured in imaging area is transmittance and/or absorptance.

The assay performed in the cavity includes but not limit to colorimetric assay, immunoassay, cell counting, cell staining, and others.

In one embodiment, the plate 1 and plate 2 are not aligned at the edge to create the flowing boundary of the sample by capillary force.

### Coating on the Sample Collection Device (a) and Sample Analyzing Device (b)

In some embodiments, the device (a) and (b)'s inner surface can have a hydrophobic coating, hydrophilic coating, or both hydrophobic and hydrophilic coatings.

In some embodiment, a coating is on at least one interior opposing surface of at least one of the plates, or both in device (b). The coating uses hydrophilic treatment, including but not limit to dielectric material coating, silicon oxide coating, plasma treatment, ozone treatment, polymer coating, acid-base treatment, surfactant chemical coating.

In some embodiments, the device's surface coating can include a hydrophobic coating.

In some embodiments, the device's surface coating can include a hydrophilic coating.

In some embodiments, the device's surface coating can include an ionic, a non-ionic, or both ionic and non-ionic coatings.

In some embodiments, the device's surface coating can include, for example, at least one of trichloro (1 H, 1 H, 2H, 2H-perfluorooctyl) silane, alkanes, oils, fats, greasy substances, or combinations thereof.

In some embodiments, to achieve the hydrophilic property inside the device, the surfactant are coated on the inner surface of the device with a final surface concentration of 1 ng/mm², 2 ng/mm², 5 ng/mm², 10 ng/mm², 20 ng/mm², 50 ng/mm², 100 ng/mm², or in a range between any two of the values.

### Reagent Coating and Staining

In some embodiments, the liquid sample for analyzing is pre-mixed with reagents before flowing into the device (a) or (b).

In some embodiments, the reagents are pre-coated on the inner surface of the device.

In some embodiments, the reagents are uniformly distributed and coated inside the device.

In some embodiments, the reagents are coated with a slope distribution inside the device to balance the final concentration in the device after adding the liquid sample.

In some embodiments, there is a slow releasing coating with the reagent inside the device. The slow releasing layer delaying the reagents dissolving into the liquid sample after the sample become static after flowing in. The slowing releasing time is for example, 3 second, 5 second, 10 second or in a range between any of these values.

The slow releasing materials comprise albumin, carbomers, carboxymethyl cellulose, carrageenan, chitosan, dextrin, polyethylene glycol, polyvinylpyrrolidone, polyvinyl alcohol, and etc.

The device, kit, system, or method of any prior embodiments, wherein the acridine orange is coated onto the first plate, or the second plate or both. The device, kit, system, or method of any prior embodiments, wherein the Zwittergent is coated onto the first plate, or the second plate or both.

The device, kit, system, or method of any prior embodiments, wherein the Methylene blue and Zwittergent is coated onto the first plate, or the second plate or both. The device, kit, system, or method of any prior embodiments, wherein the acridine orange and Zwittergent is coated onto the first plate, or the second plate or both. The device, kit, system, or method of any prior embodiments, wherein the YOYO dye and Zwittergent is coated onto the first plate, or the second plate or both.

The devices or methods of any prior embodiment, wherein the device further comprises, on one or both plates, multi reagent layers including anti-conglutination, cell lysing, cell staining, release time control material layers, and their combinations;

wherein each layer coated on the plates has a thickness of 10 nm, 100 nm, 200 nm, 500 nm, 1 μm or a range between any two of the values.

In some embodiment, to achieve a uniform distribution of red blood cell in device, Zwittergent is coated on the plate with a preferred area concentration of 3 ng/mm², 5 ng/mm², 8 ng/mm², 12 ng/mm², 15 ng/mm², 25 ng/mm², 35 ng/mm², 50 ng/mm², 80 ng/mm², 100 ng/mm² or in a range between any of the two values.

In some embodiment, to lyse red blood cell in device, Zwittergent is coated on the plate with a preferred area concentration of 100 ng/mm², 120 ng/mm², 150 ng/mm², 180 ng/mm², 200 ng/mm², 300 ng/mm², 400 ng/mm², 500 ng/mm², 800 ng/mm², 1000 ng/mm² or in a range between any of the two values.

In some embodiment, to achieve a uniform distribution of red blood cell in device, Zwittergent is coated on the plate with a preferred final concentration in blood of 0.05 mg/mL, 0.1 mg/mL, 0.2 mg/mL, 0.3 mg/mL, 0.5 mg/mL, 0.6 mg/mL, 1.0 mg/mL, 2 mg/mL or in a range between any of the two values.

In some embodiment, to lyse red blood cell in device, Zwittergent is coated on the plate with a preferred final concentration in blood of 2 mg/mL, 3 mg/mL, 4 mg/mL, 5 mg/mL, 6 mg/mL, 7 mg/mL, 9 mg/mL, 10 mg/mL, 15 mg/mL, 25 mg/mL, 50 mg/mL, or in a range between any of the two values.

In some embodiment, to achieve a uniform distribution of red blood cell in device, Zwittergent is coated on the plate with a preferred area concentration of 3 ng/mm², 5 ng/mm², 8 ng/mm², 12 ng/mm², 15 ng/mm², 25 ng/mm², 35 ng/mm², 50 ng/mm², 80 ng/mm², 100 ng/mm² or in a range between any of the two values.

In some embodiment, to lyse red blood cell in device, Zwittergent is coated on the plate with a preferred area concentration of 100 ng/mm², 120 ng/mm², 150 ng/mm², 180 ng/mm², 200 ng/mm², 300 ng/mm², 400 ng/mm², 500 ng/mm², 800 ng/mm², 1000 ng/mm² or in a range between any of the two values.

In some embodiment, to achieve a uniform distribution of red blood cell in device, Zwittergent is coated on the plate with a preferred final concentration in blood of 0.05 mg/mL, 0.1 mg/mL, 0.2 mg/mL, 0.3 mg/mL, 0.5 mg/mL, 0.6 mg/mL, 1.0 mg/mL, 2 mg/mL or in a range between any of the two values.

In some embodiment, to lyse red blood cell in device, Zwittergent is coated on the plate with a preferred final concentration in blood of 2 mg/mL, 3 mg/mL, 4 mg/mL, 5 mg/mL, 6 mg/mL, 7 mg/mL, 9 mg/mL, 10 mg/mL, 15 mg/mL, 25 mg/mL, 50 mg/mL, or in a range between any of the two values.

The device, kit, system, or method of any prior embodiments, wherein the acridine orange is coated on the plate with an area concentration of 0.5 ng/mm², 1 ng/mm², 2 ng/mm², 3 ng/mm², 5 ng/mm², 8 ng/mm², 10 ng/mm², 15 ng/mm², 20 ng/mm², 30 ng/mm² or in a range between any of the two values.

The device, kit, system, or method of any prior embodiments, wherein the acridine orange is coated on the plate with an area concentration of 3 to 10 ng/mm² and Zwittergent is coated on the plate with an area concentration of 3 to 10 ng/mm².

The device, kit, system, or method of any prior embodiments, wherein the acridine orange is coated on the plate with an area concentration of 5 to 20 ng/mm² and Zwittergent is coated on the plate with an area concentration of 10 to 30 ng/mm².

### Spacer and Bonding in the Chamber

In some embodiments, there is spacer array between the two plates. The spacer is attached on one of the plates or both plates.

The spacers can be used as physical support of the two plates, the geometry reference, dimensional reference and optical reference in the measurements.

The lateral dimension of a spacer in each direction is the same or different. In some embodiments, the lateral dimension for each direction (x or y) is 1 nm or less, 3 nm or less, 5 nm or less, 7 nm or less, 10 nm or less, 20 nm or less, 30 nm or less, 40 nm or less, 50 nm or less, 100 nm or less, 200 nm or less, 500 nm or less, 800 nm or less, 1000 nm or less, 1 μm or less, 2 μm or less, 3 μm or less, 5 μm or less, 10 μm or less, 20 μm or less, 30 μm or less, 50 μm or less, 100 μm or less, 150 μm or less, 200 μm or less, 300 μm or less, or 500 μm or less, or in a range between any two of the values.

The period of spacer array is between 1 nm to 100 nm in one preferred embodiment, 100 nm to 500 nm in another preferred embodiment, 500 nm to 1000 nm in a separate preferred embodiment, 1 μm (i.e. 1000 nm) to 2 μm in another preferred embodiment, 2 μm to 3 μm in a separate preferred embodiment, 3 μm to 5 μm in another preferred embodiment, 5 μm to 10 μm in a separate preferred embodiment, and 10 μm to 50 μm in another preferred embodiment, 50 μm to 100 μm in a separate preferred embodiment, 100 μm to 175 μm in a separate preferred embodiment, and 175 μm to 300 μm in a separate preferred embodiment

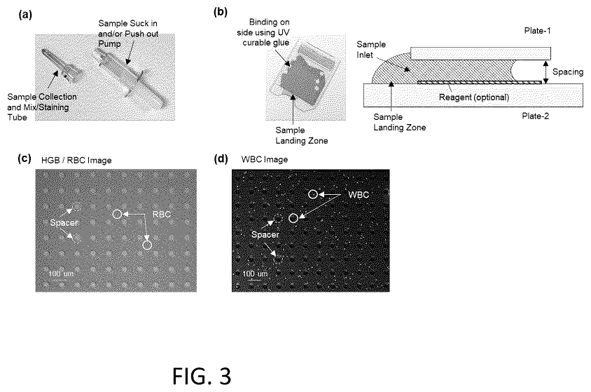

As shown in FIG. 3 of some example of the spacer insider the device, (a) the spacer are distributed in the chamber; one example is pillar array as described above; (b) Spacer is on the edge of the device. The shape can be rectangular, round or combination of them.

In some embodiment, one dimension of the spacer is 0.1 mm, 0.2 mm, 0.5 mm, 1 mm, 2 mm, 5 mm, 10 mm, 20 mm or in a range between any of the two values.

In some embodiment, the spacer is made of plastic, polymer, adhesive materials, dielectric materials.

In some embodiments, the thickness variation of the spacer is less than 1%, less than 3%, less than 5%, less than 10% or in a range between any of the two values.

In some embodiments, the spacer also functions as the adhesive structure to both plates. In this case, some of the spacer stick to both plates thus bond two plates together.

In some embodiments, there are separated bonding or adhesive structure between two chambers. The bonding structure can be paste pad, paste lines, etc. The bonding structure material can be polymer, plastic, organic materials or inorganic materials.

In some embodiments, there are separated bonding or adhesive structure outside of two chambers. The bonding structure can be clamps, paste wrap, etc. The bonding structure material can be polymer, plastic, organic materials or inorganic materials.

### Example

One example of the device is shown in FIG. 3. Examples of a set of devices for analyzing a sample, particularly blood. (a) Photo of an example sample collecting and transferring device, which is used for sample collection, sample mixing, sample staining and sample transferring. (b) Photo and structure of a sample distribution and imaging device. The device comprising: two plates oppose and bonded to each other to form the spacing, the whole blood sample can flow into the spacing between the two plates from the exterior location through sample inlet. (c) The bright field image example of RBC and HgB inside the device. (d) The fluorescence image example of stained whole blood film inside the device.

The sample collecting and transferring device, which is used for sample collection, sample mixing, sample staining and sample transferring as shown in FIG. 3A. The device has two parts connected to each other. Part (1) is a capillary tube with reagent coated inside, which is used for sample collection by capillary force from the inlet, mixture of the sample with pre-coated reagents inside the inner surface of the tube. In this example, the capillary tube is made of plastic with 10 μl volume. The reagents including dyes as Acridine orange and surfactant as Zwittergent are coated on the inner surface of the tube. The acridine orange is coated with an area concentration of 1 to 20 ng/mm² and Zwittergent is coated with an area concentration of 1 to 30 ng/mm². The volume concentration for acridine orange is between 0.2 mg/mL to 1.5 mg/mL, and for Zwittergent is between 0.2 mg/mL to 10 mg/mL. The reagent coating method can be air dye or lyophilization.

Part (2) is optional, which can be a gas pump, a pipette or a plunger, which is used for providing the external force to suck in and/or push out the sample in the Part (1). In this example, the Part (2) is a plastic mini pipette, which can be mechanically hooked to Part (1) and sealed.

FIG. 3B shows an example sample distribution and imaging device. One embodiments of the device comprising: two plates oppose to each other, wherein the opposed plates define an interior cavity; an exterior liquid sample contact area or sample inlet on an exterior location of the device, wherein the interior cavity is in fluid communication with the exterior liquid sample contact area; the liquid sample can flow into the spacing between the two plates from the exterior location through sample inlet. There is a sample landing Zone at the exterior location of the device which connected to the sample inlet. Optionally, there are reagents pre-coated inside the cavity. In this example: One of the plates has a thickness of 200 μm to 1500 μm. The other plate has a thickness of 50 μm to 250 μm. There is pillar array on the plate 2 with a pillar height from 20 μm to 40 μm with a inter pillar distance of 100 μm to 200 μm and a pillar size around 10 μm to 40 μm. The pillar array can also be fabricated on the plate 1. The two plates are bonded near the edge of the device with UV curable polymer as shown in FIG. 3B.

The blood can be flowed into the device from the inlet at one edge of the device without binding material. The staining reagents can be pre-mixed with blood before flowing in. The staining reagent can also be pre-coated on the plates insider the device.

In some embodiments, the plate surface coating can include a hydrophilic coating.

In some embodiments, the plate surface coating includes a surfactant, as Zwittergent, with a concentration between 1 to 500 ng/mm².

In some embodiments, the plate surface coating can include an ionic, a non-ionic, or both ionic and non-ionic coatings.

In some embodiments, the cell lysing agent comprises ammonium chloride, sodium bicarbonate, ethylenediaminetetraacetic acid (EDTA), acetic acid, citric acid, or other acid and base, or any combinations thereof.

The acridine orange or other staining reagents is coated onto the first plate, or the second plate or both.

The Zwittergent or other detergent is coated onto the first plate, or the second plate or both.

The acridine orange is coated on the plate with an area concentration of 1 to 20 ng/mm² and Zwittergent is coated on the plate with an area concentration of 1 to 30 ng/mm².

From the testing results, the stained whole blood is uniformly distributed inside the chamber as shown in FIG. 3C. The labeled areas are spacer and whole blood. The WBC in the sample are stained by the fluorescence dye in the sample collection device and labeled in FIG. 3D. Using the computer vision algorithm, the RBC, HGB and WBC numbers can be analyzed and counted. By dividing the cell number by the volume of the analyzing area, the concentration of the analytes in the sample can be accurately measured.

In one embodiment, the reagent coated on the sample collection device includes surfactant as Zwittergent, anti-coagulation reagents as K₂EDTA, fluorescence dye as Acridine orange, colorimetric dye as methylene blue.

In some embodiments, the surfactant comprises Zwittergent, ASB-14, ASB-16, CHAPS, Cationic surfactant IIa, IIb, Ic, IId, CTAC, Tween 20, Tween 40, Tween 60, Tween 80, Saponin, Sodium lauryl sulfate (SLS), ammonium lauryl sulfate, CTAB, sodium lauryl ether sulfate (SLES), sodium myreth sulfate, docusate, perfluorooctanesulfonate, alkyl-aryl ether phosphates, alkyl ether phosphates, CTAB, cetylpyridinium chloride (CPC), benzalkonium chloride (BAC), benzethonium chloride (BZT), dimethyldioctadecylammonium chloride, dioctadecyldimethlyammonium bromide (DODAB), cocamidopropyl hydroxysultaine, cocamidopropyl betaine, narrow-range ethoxylate, octaethylene glycol monododecyl ether, pentaethylene glycol monododecyl ether, nonxynols, Triton X-100, polyethoxylated tallow amine, cocamide monoethanolamine, cocamide diethanolamine, poloxamers, glycerol monostearate, glycerol monolaurate, sorbitan monolaurate, sorbitan monostearate, sorbitan tristearate, decyl glucoside, lauryl glucoside, octyl glucoside, lauryldimethylamine oxide, dimethyl sulfoxide, phosphine oxide.

In some embodiments, the cell stain agent comprises Wright's stain (Eosin, methylene blue), Giemsa stain (Eosin, methylene blue, and Azure B), May-Grunwald stain, Leishman's stain (“Polychromed” methylene blue (i.e. demethylated into various azures) and eosin), Erythrosine B stain (Erythrosin B), and other fluorescence stain including but not limit to Acridine orange dye, Acridine red dye, 3,3-dihexyloxacarbocyanine (DiOC6), Propidium Iodide (PI), Fluorescein Isothiocyanate (FITC) and Basic Orange 21 (B021) dye, Ethidium Bromide, Brilliant Sulfaflavine and a Stilbene Disulfonic Acid derivative, Erythrosine B or trypan blue, Hoechst 33342, Trihydrochloride, Trihydrate, or DAPI (4′,6-Diamidino-2-Phenylindole, Dihydrochloride), or any combinations thereof.

In some embodiment, the surfactant as Zwittergent is filled inside the tube first, then dried or lyophilized for coating. The filled solution concentration for Zwittergent is 0.1 mg/mL, 0.2 mg/mL, 0.5 mg/mL, 0.6 mg/mL, 1.0 mg/mL, 2 mg/mL, 3 mg/mL, 4 mg/mL, 5 mg/mL, 6 mg/mL or in a range between any of the two values. The filled solution concentration for Zwittergent us 1.0 mg/mL, 5 mg/mL, 7 mg/mL, 10 mg/mL, 15 mg/mL, 20 mg/mL, 50 mg/mL or in a range between any of the two values. The preferred concentration for Zwittergent is 3 mg/mL, 5 mg/mL, 6 mg/mL, 7 mg/mL, 8 mg/mL, or in a range between any of the two values.

In some embodiment, the fluorescence dye as Acridine orange is filled inside the tube first, then dried or lyophilized for coating. The filled solution concentration for Acridine orange is 0.1 mg/mL, 0.2 mg/mL, 0.5 mg/mL, 0.6 mg/mL, 1.0 mg/mL, 1.5 mg/mL, 2 mg/mL, 3 mg/mL, 5 mg/mL, 10 mg/mL or in a range between any of the two values. The preferred filled solution concentration for Acridine orange is 0.5 mg/mL, 0.6 mg/mL, 1.0 mg/mL, 1.5 mg/mL, 2.0 mg/mL, or in a range between any of the two values.

In some embodiment, the anti-coagulation reagents as K2EDTA is filled inside the tube first, then dried or lyophilized for coating. The filled solution concentration for K2EDTA is 0.2 mg/mL, 0.5 mg/mL, 1.0 mg/mL, 1.5 mg/mL, 2.0 mg/mL, 3.0 mg/mL, 5 mg/mL, 10 mg/mL or in a range between any of the two values. The preferred filled solution concentration for Acridine orange is 0.5 mg/mL, 1.0 mg/mL, 1.5 mg/mL, 1.8 mg/mL, 2.0 mg/mL, or in a range between any of the two values.

In some embodiment, to lyse red blood cell in device, Zwittergent is coated on the tube with a preferred final concentration in blood, after blood sucking into the tube, of 2 mg/mL, 3 mg/mL, 4 mg/mL, 5 mg/mL, 6 mg/mL, 7 mg/mL, 9 mg/mL, 10 mg/mL, 15 mg/mL, 25 mg/mL, 50 mg/mL, or in a range between any of the two values, with a preferred range of 3 mg/mL to 8 mg/mL.

In some embodiment, to achieve a uniform distribution of red blood cell in device, Zwittergent is coated on the tube with a preferred final concentration in blood, after blood sucking into the tube, of 0.3 mg/mL, 0.5 mg/mL, 0.8 mg/mL, 1.0 mg/mL, 1.2 mg/mL, 1.5 mg/mL, 2 mg/mL, 3 mg/mL, or in a range between any of the two values, with a preferred range of 0.5 mg/mL to 2.0 mg/mL.

In some embodiment, to achieve fluorescence staining of white blood cell and platelets in device, Acridine orange is coated on the tube with a preferred final concentration in blood, after blood sucking into the tube, of 0.3 mg/mL, 0.5 mg/mL, 0.8 mg/mL, 1.0 mg/mL, 1.2 mg/mL, 1.5 mg/mL, 2 mg/mL, or in a range between any of the two values, with a preferred range of 0.5 mg/mL to 2.0 mg/mL.

### Device for Improving a Mixing of a Sample and a Reagent and the Method of Using the Same.

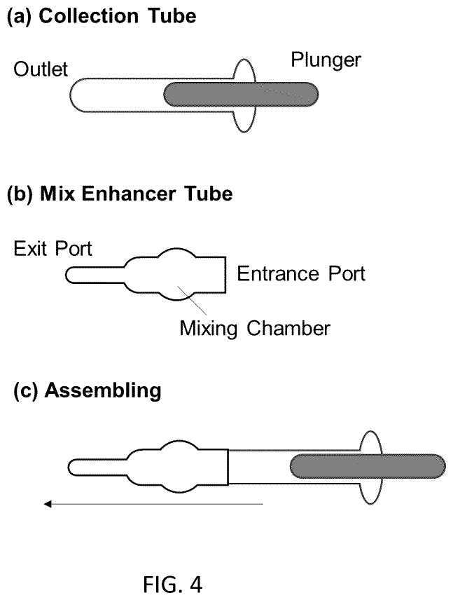

A-3 According to the present invention, as illustrated in FIG. 4, a method of improving a mixing of a sample and a reagent, comprising:
   obtaining a sample collector the comprises:
      (i) a capillary tube that has a reagent inside the tube and suck a liquid sample from outside the tube into the inside of the tube using capillary force; and
      (ii) a plunger that can push the sample sucked inside the capillary tube outside of the tube; and
   obtaining a mixing enhancing tube that has a mixing chamber that further mix the sample and the reagent; wherein the mixing chamber has the structures that cause the sample and the reagent enter the chamber mix further;
   collecting the sample using the sample collector;
   connecting an entrance port of the mixing enhancing tube with the exit port of the collecting tube;
   pushing, using the plunger, the sample in the sample collection tube out the exit port of the collection tube, and making the sample (a) entering the enhance port of the mixing enhancing tube, (b) entering the mixing chamber, and (c) exiting from the exit port of the mixing enhancing tube.
A-4 According to the present invention, as illustrated in FIG. 4, a kit of improving a mixing of a sample with a reagent, comprising:
   a sample collector the comprises:
      (i) a capillary tube that has a reagent inside the tube and sucks a liquid sample from outside the tube into the inside of the tube using capillary force; and
      (ii) a plunger that can push the sample sucked inside the capillary tube to outside of the tube; and a mixing enhancing tube comprising
      (i) a mixing chamber that further mix the sample and the reagent that enters the mixing chamber; wherein the mixing chamber has the structures that cause the sample and the reagent enter the chamber mix further;
      (ii) The mixing chamber has an entrance and an exit, wherein the entrancing port that connects to the exit of the collection tube and that allow the sample entering into the mixing chamber, and the exit port that allow the sample from the mixing chamber out of the mixing enhancing tube.

In some embodiments, the mixing chamber comprises microfluidic structures that improve the sample and reagent mixing.

In some embodiments, the mixing chamber comprises microfluidic structures that generate a turbulence in the sample flow to improve a mixing of the sample and reagent that flow through the mixing chamber.
A-5 According to the present invention, as illustrated in FIG. 4, a method of improving a mixing of a sample and a reagent, comprising:
   obtaining the sample collector in embodiment A-4;
   obtaining the mixing enhancing tube in embodiment A-4 collecting the sample using the sample collector;
   pushing, using the plunger, the sample in the sample collection tube out the exit port of the collection tube, and making the sample (a) entering the enhance port of the mixing enhancing tube, (b) entering the mixing chamber, and (c) exiting from the exit port of the mixing enhancing tube.

In some embodiments, the sample mixed with an reagent further is deposited on a sample holder with two plates separated by a small gap. The sample is further suched into the spacing between the two plates of the sample holder, and the sample was imaged and/or measured while the sample is in the sample holder. A flow of the sample into the sample holder can further improve a mixing between the sample and the reagent.

It is appreciated that the device, system, and method in this disclosure may apply to various liquid samples, including a blood sample, with or without apparent modification. Such modification should be understood as being within the scope of this disclosure.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The term “a,” “an,” or “the” cover both the singular and the plural reference, unless the context clearly dictates otherwise. The terms “comprise,” “have,” “include,” and “contain” are open-ended terms, which means “include but not limited to,” unless otherwise indicated.

The “substantially uniform thickness” means a thickness that is constant or only fluctuates around a mean value, for example, by no more than 10%, and preferably no more than 5%.

The term “and/or” means any one or more of the items in the list joined by “and/or.” As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y, and/or z” means “one or more of x, y, and z.”

### Aspects:

Any of Aspects 1-49 are combinable with any of Aspects 50-51.
1. A method of preparing and analyzing a whole blood with rapid simple sample preparation steps, without dilution, and with improved reagent mixing uniformity, improved cell distribution in blood, and reduced cell aggregation, comprising:
   obtaining a sample collector;
   obtaining a sample analyzing card;
   pricking skin of a subject to cause a bleeding (e.g., prick a finger);
   collecting the blood from the skin using the sample collector;
   depositing, after collecting the whole blood sample, without diluting the sample, without shaking the sample, within in 3 (three) min after the sample collection, the blood sample at the inlet of a lateral microfluidic channel the analyzing card; wherein the analyzing card sucks at least a part of the sample inside of the card; and
   analyzing the at least part of the blood sample inside of the card;
   wherein the sample collector comprising:
      (iv) a capillary tube that has a reagent inside the tube and suck a liquid sample from outside the tube into the inside of the tube using capillary force;
      (v) a plunger that can push the sample sucked inside the capillary tube outside of the tube; and
      (vi) the reagent comprises zwittergent of a concentration selected in the range of 0.2 mg/mL to 10 mg/mL, acridine orange of a concentration selected in the range of 0.2 mg/mL to 3 mg/mL, or any combination for improving the cell distribution in the blood with less aggregation and for staining the cell; and
   wherein the analyzing card comprises two plates forming the lateral microfluidic channel that has a channel height of 200 μm or less.
2 A kit of preparing and analyzing a whole blood with rapid simple sample preparation steps, without dilution, and with improved reagent mixing uniformity, improved cell distribution in blood, and reduced cell aggregation, comprising:
   a sample collector; and
   a sample analyzing card;
   wherein the sample collector comprising:
      (vi) a capillary tube that has a reagent inside the tube and suck a liquid sample from outside the tube into the inside of the tube using capillary force;
      (vii) a plunger that can push the sample sucked inside the capillary tube outside of the tube; and
      (viii) the reagent comprises zwittergent of a concentration selected in the range of 0.2 mg/mL to 10 mg/mL, acridine orange of a concentration selected in the range of 0.2 mg/mL to 3 mg/mL, or any combination for improving the cell distribution in the blood with less aggregation and for staining the cell; and
   wherein the analyzing card comprises two plates forming the lateral microfluidic channel that has a channel height of 200 μm or less.
3. The method of Aspect 1 and the kit of Aspect 2, wherein the sample analyzing card further comprising a reagent on one or both of the two plates.
4. The method or kit of any of Aspects 1-3, wherein the channel height is 30 μm.
5. The method or kit of any of Aspects 1-4, wherein the channel height is 10 μm or less.
6. The method or kit of any of Aspects 1-5, wherein the channel height is 5 μm.
7. The method or kit of any of Aspects 1-6, wherein the card has multiple of lateral flow microfluidic channels on sample analyzing card.
8. The method or kit of any of Aspects 1-7, wherein the card has multiple of lateral flow microfluidic channels on sample analyzing card, wherein the channel of different channel height of 5 μm and 30 μm.
9. The method or kit of any of Aspects 1-8, wherein the card has multiple of lateral flow microfluidic channels on sample analyzing card, wherein at least a first channel has channel height in range of 3 μm to 10 μm, and at least a second channel has a channel height in range of 20 μm to 40 μm.
10. The method or kit of any of Aspects 1-9, wherein a concentration of zwittergent is in the range of 0.2 mg/mL to 10 mg/mL, a concentration of acridine orange is in the range of 0.2 mg/mL to 3 mg/m, or any combinations.
11. The method or kit of any of Aspects 1-9, wherein a concentration of zwittergent is in the range of 0.5 mg/mL to 6 mg/mL, a concentration of acridine orange is in the range of 0.5 mg/mL to 2 mg/m, or any combinations.
12. The method or kit of any of Aspects 1-9, wherein a concentration of zwittergent is in the range of 1 mg/mL to 5 mg/mL, a concentration of acridine orange is in the range of 0.6 mg/mL to 1.5 mg/m, or any combinations.
13. The method or kit of any of Aspects 1-9, wherein a concentration of zwittergent is in the range of 1 mg/mL to 2 mg/mL, the concentration of acridine orange is in the range of 0.6 mg/mL to 1.5 mg/m, or any combinations.
14. The method or kit of any of Aspects 1-9, wherein a concentration of zwittergent is in the range of 4 mg/mL to 6 mg/mL, a concentration of acridine orange is in the range of 0.6 mg/mL to 1.5 mg/m, or any combinations.
15. The method or kit of any of Aspects 1-14, wherein the sample is deposited from the collection tube on the sample analyzing card within in 2 (two) min after the sample collection.
16. The method or kit of any of Aspects 1-15, wherein the sample is deposited from the collection tube on the sample analyzing card within in 1 (one) min after the sample collection.
17. A kit for making a sample that flow inside of a lateral flow microfluidic channel with a channel height of 100 micron or less and a width of 200 μm or wider, comprising: a lateral flow microfluidic channel and a sample collection tube

Wherein the lateral flow microfluidic channel comprising:
(iv) a channel that has a height of 100 micron or less and a width of 200 μm or wider;
(v) an sample inlet of the channel,
(vi) a sample landing area next to the inlet wherein the sample collection tube comprising:
(ix) a capillary tube that has reagent inside of the tube and can suck a liquid sample outside of the tube into the tube using capillary force;
(x) a plunger that can push the sample sucked inside the capillary tube outside of the tube;
18. A kit for analyzing a sample, particularly blood, comprising:
   (i) a sample collecting and transferring device (device (a)), which is used for sample collection, sample mixing, sample staining and sample transferring, wherein the device (a) comprises part (1) and an optional part (2) connected to each other, the part (1) is a capillary tube with reagent coated inside, which is used for sample collection from the inlet, mixture of the sample with pre-coated reagents inside the inner surface of the tube, the part (2) is optional is used for providing an external force to suck in and/or push out the sample in the part (1);
   (i) a sample distribution and imaging device (device (b)) comprising: two plates oppose to each other, wherein the opposing plates define an interior cavity; an exterior liquid sample contact area or sample inlet on an exterior location of the device, wherein the interior cavity is in fluid communication with the exterior liquid sample contact area, the liquid sample is capable of flowing into the spacing between the two plates from the exterior location through sample inlet, a sample landing Zone at the exterior location of the device is connected to the sample inlet.
19. The kit of Aspect 18, wherein there are reagents pre-coated inside the cavity.
20. A method of performing biological and chemical assays using the kit of claim 18, comprising the steps:
   (1) collecting sample as whole blood into a sample collecting and transferring device, the sample are mixed and stained by the reagents in the device and dropping the sample onto the exterior liquid sample contact area;
   (2) transferring the sample from the device by providing the external force to push out the sample to the sample landing Zone at the exterior location of a sample distribution and imaging device;
   (3) flowing the sample into the cavity of the sample distribution and imaging device from the sample landing Zone and mix with reagent inside the sample distribution and imaging device and distribute inside the sample distribution and imaging device; and
   (4) imaging and analyzing the sample inside the sample distribution and imaging device.
21. The method of Aspect 20, wherein the liquid sample is whole blood, and the analyte to be analyzed is red blood cells, white blood cells and platelets.
22. The method of any of Aspects 20-21, wherein parameters measured in an imaging area is complete blood count, red blood cell count, hemoglobin, platelet count, white blood cell differentiation and count, and/or abnormal cell types.
23. The kit or method of any of Aspects 18-24, wherein the reagent coated on the sample collecting and transferring device is surfactant as Zwittergent, anti-coagulation reagents as K₂EDTA.
24. The kit or method of any of Aspects 18-23, wherein a volume of the sample collecting and transferring device is 1 μl, 3 μl, 5 μl, 10 μl, 15 μl, 20 μl or in a range between any of these values.
25. The kit or method of any of Aspects 18-24, wherein the spacing height between two plates is 2 μm, 3 μm, 5 μm, 8 μm, 10 μm or in a range between any of these values.
26. The kit or method of any of Aspects 18-24, wherein the spacing height between two plates is 4 μm, 5 μm, 6 μm, or in a range between any of these values.
27. The kit or method of any of Aspects 18-25, wherein the reagent coated on the collecting and transferring device is surfactant as Zwittergent, anti-coagulation reagents as K₂EDTA.
28. The kit or method of any of Aspects 18-24, wherein the spacing height between two plates is 10 μm, 15 μm, 20 μm, 30 μm, 50 μm, 80 μm, 100 μm, 150 μm or in a range between any of these values;
29. The kit or method of any of Aspects 18-24, wherein the preferred spacing height between two plates is 30 μm, 50 μm, 80 μm, 100 μm, 150 μm or in a range between any of these values.
30. The kit or method of any of Aspects 18-29, wherein the reagent coated on the collecting and transferring device is surfactant as Zwittergent, anti-coagulation reagents as K₂EDTA, fluorescence dye as Acridine orange, colorimetric dye as methylene blue.
31. The kit or method of any of Aspects 18-29, wherein the reagent coated on the collecting and transferring device is surfactant as Zwittergent, anti-coagulation reagents as K₂EDTA, fluorescence dye as Acridine orange and YOYO, colorimetric dye as methylene blue.
32. The kit or method of any of Aspects 18-31, wherein the volume of the sample collection device is 1 μl, 3 μl, 5 μl, 10 μl, 15 μl, 20 μl or in a range between any of these values.
33. The kit or method of any of Aspects 18-32, wherein the spacing height between two plates is 1 μm, 5 μm, 8 μm, 10 μm, 15 μm, 20 μm, 30 μm, or in a range between any of these values.
34. The kit or method of any of Aspects 18-33, wherein the spacing height between two plates is 1 μm, 2 μm, 5 μm, 10 μm, 15 μm, 20 μm, or in a range between any of these values.
35. The kit or method of any of Aspects 18-34, wherein there are several different spacing areas in the chamber.
36. The kit or method of Aspect 35, wherein the spacing of each area is 1 μm, 2 μm, 3 μm, 5 μm, 10 μm, 20 μm, 30 μm, 50 μm, 100 μm, 150 μm or in a range between any two of the values.
37. The kit or method of any of Aspects 35-36, wherein the spacing of one area is 2 μm, 3 μm, 5 μm, 10 μm, or in a range between any two of the values.
38. The kit or method of any of Aspects 35-36, wherein the preferred spacing of one area is 10 μm, 30 μm, 50 μm, 100 μm, or in a range between any two of the values.
39. The kit or method of any of Aspects 35-38, wherein the difference between two spacing of each area is 0.5 μm, 1 μm, 2 μm, 3 μm, 5 μm, 10 μm, 20 μm, 30 μm, 50 μm, 100 μm, 150 μm, or in a range between any two of the values.
40. The kit or method of any of Aspects 35-38, wherein the ratio of the manufacturing spacing height between two area is 1.1 fold, 1.2 fold, 1.5 fold, 2 fold, 3 fold, 5 fold, 10 fold, 30 fold, 50 fold, 100 fold, or in a range between any two of the values.
41. The kit or method of any of Aspects 18-40, wherein the signal measured in imaging area is cell or particle numbers.
42. The kit or method of any of Aspects 18-40, wherein the signal measured in imaging area is colorimetric intensity.
43. The kit or method of any of Aspects 18-40, wherein the signal measured in imaging area is transmitted light intensity.
44. The kit or method of any of Aspects 18-40, wherein the signal measured in imaging area is fluorescence signal intensity.
45. The kit or method of any of Aspects 18-40, wherein the signal measured in imaging area is transmittance and/or absorptance.
46. The kit or method of any of Aspects 18-40, wherein the plate 1 and plate 2 are not aligned at the edge to create the flowing boundary of the sample by capillary force.
47. The kit or method of any of Aspects 18-45, wherein the sample collecting and transferring device and the sample distribution and imaging device each comprises an inner surface having a hydrophobic coating, hydrophilic coating, or both hydrophobic and hydrophilic coatings.
48. The kit or method of any of Aspects 18-45, wherein a coating is on at least one interior opposing surface of at least one of the plates, or both in sample distribution and imaging device.
49. The kit or method of any Aspects 18-46, wherein a surface coating of the devices includes a hydrophobic coating.
50. The kit or method of any Aspects 18-46, wherein a surface coating of the devices includes a hydrophilic coating.
51. The kit or method of any Aspects 18-46, wherein a surface coating of the devices includes an ionic, a non-ionic, or both ionic and non-ionic coatings.
52. The kit or method of any Aspects 18-46, wherein a surface coating of the devices includes at least one of trichloro (1 H, 1 H, 2H, 2H-perfluorooctyl) silane, alkanes, oils, fats, greasy substances, or combinations thereof.
53. The kit or method of any Aspects 18-52, wherein the liquid sample for analyzing is pre-mixed with reagents before flowing into the device (a) or (b).
54. The kit or method of any Aspects 18-52, wherein the reagents are pre-coated on the inner surface of the device.
55. The kit or method of any Aspects 18-53, wherein the reagents are uniformly distributed and coated inside the device.
56. The kit or method of any Aspects 18-53, wherein the reagents are coated with a slope distribution inside the device to balance the final concentration in the device after adding the liquid sample.
57. The kit or method of any Aspects 18-53, wherein the reagents, wherein there is a slow releasing coating with the reagent inside the device, the slow releasing layer delaying the reagents dissolving into the liquid sample after the sample become static after flowing in.
58. The kit or method of Aspect 57, wherein the slowing releasing time is 3 second, 5 second, 10 second or in a range between any of these values.
59. The kit or method of any of Aspects 57-58, wherein the slow releasing materials comprise albumin, carbomers, carboxymethyl cellulose, carrageenan, chitosan, dextrin, polyethylene glycol, polyvinylpyrrolidone, and/or polyvinyl alcohol.
60. The kit or method of any of Aspects 1-59, wherein the acridine orange is coated onto the first plate, or the second plate or both.
61. The kit or method of any of Aspects 1-59, wherein the Zwittergent is coated onto the first plate, or the second plate or both.
62. The kit or method of any of Aspects 1-59, wherein the Methylene blue and Zwittergent is coated onto the first plate, or the second plate or both.
63. The kit or method of any of Aspects 1-59, wherein the acridine orange and Zwittergent is coated onto the first plate, or the second plate or both.
64. The kit or method of any of Aspects 1-59, wherein the YOYO dye and Zwittergent is coated onto the first plate, or the second plate or both.
65. The kit or method of any of Aspects 1-64, wherein the device further comprises, on one or both plates, multi reagent layers including anti-conglutination, cell lysing, cell staining, release time control material layers, and their combinations, wherein each layer coated on the plates has a thickness of 10 nm, 100 nm, 200 nm, 500 nm, 1 μm or a range between any two of the values.
66. The kit or method of any of Aspects 1-65, wherein, to achieve a uniform distribution of red blood cell in device, Zwittergent is coated on the plate with an area concentration of 3 ng/mm², 5 ng/mm², 8 ng/mm², 12 ng/mm², 15 ng/mm², 25 ng/mm², 35 ng/mm², 50 ng/mm², 80 ng/mm², 100 ng/mm² or in a range between any of the two values.
67. The kit or method of any of Aspects 1-65, wherein, to lyse red blood cell in device, Zwittergent is coated on the plate with a preferred area concentration of 100 ng/mm², 120 ng/mm², 150 ng/mm², 180 ng/mm², 200 ng/mm², 300 ng/mm², 400 ng/mm², 500 ng/mm², 800 ng/mm², 1000 ng/mm² or in a range between any of the two values.
68. The kit or method of any of Aspects 1-65, wherein, to achieve a uniform distribution of red blood cell in device, Zwittergent is coated on the plate with a preferred final concentration in blood of 0.05 mg/mL, 0.1 mg/mL, 0.2 mg/mL, 0.3 mg/mL, 0.5 mg/mL, 0.6 mg/mL, 1.0 mg/mL, 2 mg/mL or in a range between any of the two values.
69. The kit or method of any of Aspects 1-65, wherein, to lyse red blood cell in device, Zwittergent is coated on the plate with a preferred final concentration in blood of 2 mg/mL, 3 mg/mL, 4 mg/mL, 5 mg/mL, 6 mg/mL, 7 mg/mL, 9 mg/mL, 10 mg/mL, 15 mg/mL, 25 mg/mL, 50 mg/mL, or in a range between any of the two values.
70. The kit or method of any of Aspects 1-65, wherein, to achieve a uniform distribution of red blood cell in device, Zwittergent is coated on the plate with a preferred area concentration of 3 ng/mm², 5 ng/mm², 8 ng/mm², 12 ng/mm², 15 ng/mm², 25 ng/mm², 35 ng/mm², 50 ng/mm², 80 ng/mm², 100 ng/mm² or in a range between any of the two values.
71. The kit or method of any of Aspects 1-65, wherein, to lyse red blood cell in device, Zwittergent is coated on the plate with a preferred area concentration of 100 ng/mm², 120 ng/mm², 150 ng/mm², 180 ng/mm², 200 ng/mm², 300 ng/mm², 400 ng/mm², 500 ng/mm², 800 ng/mm², 1000 ng/mm² or in a range between any of the two values.
72. The kit or method of any of Aspects 1-65, wherein, to achieve a uniform distribution of red blood cell in device, Zwittergent is coated on the plate with a preferred final concentration in blood of 0.05 mg/mL, 0.1 mg/mL, 0.2 mg/mL, 0.3 mg/mL, 0.5 mg/mL, 0.6 mg/mL, 1.0 mg/mL, 2 mg/mL or in a range between any of the two values.
73. The kit or method of any of Aspects 1-65, wherein, to lyse red blood cell in device, Zwittergent is coated on the plate with a preferred final concentration in blood of 2 mg/mL, 3 mg/mL, 4 mg/mL, 5 mg/mL, 6 mg/mL, 7 mg/mL, 9 mg/mL, 10 mg/mL, 15 mg/mL, 25 mg/mL, 50 mg/mL, or in a range between any of the two values.
74. The kit or method of any of Aspects 1-65, wherein, the acridine orange is coated on the plate with an area concentration of 0.5 ng/mm², 1 ng/mm², 2 ng/mm², 3 ng/mm², 5 ng/mm², 8 ng/mm², ng/mm², 15 ng/mm², 20 ng/mm², 30 ng/mm² or in a range between any of the two values.
75. The kit or method of any of Aspects 1-65, wherein, the acridine orange is coated on the plate with an area concentration of 3 to 10 ng/mm² and Zwittergent is coated on the plate with an area concentration of 3 to 10 ng/mm².
76. The kit or method of any of Aspects 1-65, wherein, the acridine orange is coated on the plate with an area concentration of 5 to 20 ng/mm² and Zwittergent is coated on the plate with an area concentration of 10 to 30 ng/mm².
77. The kit or method of any of Aspects 1-76, there is spacer array between the two plates.
78. The kit or method of Aspect 77, wherein the spacers are attached on one of the plates or both plates, wherein the spacers can be used as physical support of the two plates, the geometry reference, dimensional reference, and/or optical reference in the measurements.
79. The kit or method of any of Aspects 77-78, wherein the lateral dimension of a spacer in each direction is the same or different.
80. The kit or method of any of Aspects 77-79, wherein the lateral dimension for each direction (x or y) is 1 nm or less, 3 nm or less, 5 nm or less, 7 nm or less, 10 nm or less, 20 nm or less, 30 nm or less, 40 nm or less, 50 nm or less, 100 nm or less, 200 nm or less, 500 nm or less, 800 nm or less, 1000 nm or less, 1 μm or less, 2 μm or less, 3 μm or less, 5 μm or less, 10 μm or less, 20 μm or less, 30 μm or less, 50 μm or less, 100 μm or less, 150 μm or less, 200 μm or less, 300 μm or less, or 500 μm or less, or in a range between any two of the values.
81. The kit or method of any of Aspects 77-79, wherein the period of spacer array is between 1 nm to 300 μm.
82. The kit or method of any of Aspects 77-81, wherein one dimension of the spacer is 0.1 mm, 0.2 mm, 0.5 mm, 1 mm, 2 mm, 5 mm, 10 mm, 20 mm or in a range between any of the two values.
83. The kit or method of any of Aspects 77-82, wherein the spacer is made of plastic, polymer, adhesive materials, dielectric materials.
84. The kit or method of any of Aspects 77-83, wherein the thickness variation of the spacer is less than 1%, less than 3%, less than 5%, less than 10% or in a range between any of the two values.
85. The kit or method of any of Aspects 77-83, wherein the spacers function as the adhesive structure to both plates.
86. The kit or method of any of Aspects 77-85, wherein there are separated bonding or adhesive structure between two chambers.
87. The kit or method of Aspect 86, wherein the bonding structure is a paste pad or paste lines.
88. The kit or method of Aspect 86, wherein the bonding structure material is a polymer, plastic, organic materials or inorganic materials.
89. The kit or method of Aspects 77-88, wherein there are separated bonding or adhesive structure outside of two chambers. The bonding structure can be clamps, paste wrap, etc. The bonding structure material can be polymer, plastic, organic materials or inorganic materials.
90. A method of improving a mixing of a sample and a reagent, comprising:
   obtaining a sample collector the comprises:
      (i) a capillary tube that has a reagent inside the tube and suck a liquid sample from outside the tube into the inside of the tube using capillary force; and
      (ii) a plunger that can push the sample sucked inside the capillary tube outside of the tube; and obtaining a mixing enhancing tube that has a mixing chamber that further mix the sample and the reagent; wherein the mixing chamber has the structures that cause the sample and the reagent enter the chamber mix further;
   collecting the sample using the sample collector;
   connecting an entrance port of the mixing enhancing tube with the exit port of the collecting tube;
   pushing, using the plunger, the sample in the sample collection tube out the exit port of the collection tube, and making the sample (a) entering the enhance port of the mixing enhancing tube, (b) entering the mixing chamber, and (c) exiting from the exit port of the mixing enhancing tube.
91. A kit of improving a mixing of a sample and a reagent, comprising:
   a sample collector the comprises:
      (iii) a capillary tube that has a reagent inside the tube and suck a liquid sample from outside the tube into the inside of the tube using capillary force; and
      (iv) a plunger that can push the sample sucked inside the capillary tube outside of the tube; and
   a mixing enhancing tube comprising
      (iii) a mixing chamber that further mix the sample and the reagent; wherein the mixing chamber has the structures that cause the sample and the reagent enter the chamber mix further;
      (iv) an entrancing port that connects to the exit of the collection tube and that allow the sample entering into the mixing chamber;
      (v) an exit port that allow the sample from the mixing chamber out of the mixing enhancing tube.

In some embodiments, the mixing chamber comprises microfluidic structures that improve the sample and reagent mixing.

In some embodiments, the mixing chamber comprises microfluidic structures that improve the sample and reagent mixing through a turbulence of the sample flow.

We claim:

1. A kit for collection, mixing, staining, transferring, imaging, and analyzing a sample, comprising:
(i) a sample collecting and transferring device (device (a)), which is used for sample collection, sample mixing, sample staining and sample transferring, wherein the device (a) comprises part (1) and an optional part (2) connected to each other, the part (1) is a capillary tube with an reagent coated inside, which is used for sample collection from the inlet, mixture of the sample with pre-coated reagents inside the inner surface of the tube, the part (2) is optional is used for providing an external force to suck in and/or push out the sample in the part (1);
(ii) a sample distribution and imaging device (device (b)) comprising:
two plates opposing to each other, wherein the two plates define an interior cavity; and
an exterior liquid sample contact area or sample inlet on an exterior location of the device (b),
wherein the interior cavity is in fluid communication with the exterior liquid sample contact area, the liquid sample is capable of flowing into the spacing between the two plates from the exterior location through sample inlet and a sample landing Zone that is at the exterior location of the device (b) and connected to the sample inlet.

2. The kit of claim 1, wherein a reagent pre-coated inside the cavity.

3. The kit of claim 1, wherein the reagent coated on the sample collecting and transferring device is surfactant as Zwittergent, anti-coagulation reagents as K₂EDTA.

4. The kit of claim 1, wherein a volume of the sample collecting and transferring device is 1 μl, 3 μl, 5 μl, 10 μl, 15 μl, 20 μl or in a range between any of these values.

5. The kit of claim 1, wherein the spacing height between the two plates is 2 μm, 3 μm, 5 μm, 8 μm, 10 μm or in a range between any of these values.

6. The kit of claim 1, wherein the reagent coated on the collecting and transferring device is surfactant as Zwittergent, anti-coagulation reagents as K₂EDTA.

7. The kit of claim 1, wherein the spacing height between two plates is 10 μm, 15 μm, 20 μm, 30 μm, 50 μm, 80 μm, 100 μm, 150 μm or in a range between any of these values.

8. The kit of claim 1, wherein the preferred spacing height between two plates is 30 μm, 50 μm, 80 μm, 100 μm, 150 μm or in a range between any of these values.

9. The kit of claim 1, wherein the reagent coated on the collecting and transferring device is surfactant as Zwittergent, anti-coagulation reagent as K₂EDTA, fluorescence dye as Acridine orange, colorimetric dye as methylene blue.

10. The kit of claim 1, wherein the reagent coated on the collecting and transferring device is surfactant as Zwittergent, anti-coagulation reagent as K₂EDTA, fluorescence dye as Acridine orange and YOYO, colorimetric dye as methylene blue.

11. The kit or method of claim 1, wherein the volume of the sample collection device is 1 μl, 3 μl, 5 μl, 10 μl, 15 μl, 20 μl or in a range between any of these values.

12. The kit of claim 1, wherein the spacing height between two plates is 1 μm, 5 μm, 8 μm, 10 μm, 15 μm, 20 μm, 30 μm, or in a range between any of these values.

13. The kit of claim 1, wherein there are several different spacing areas in the chamber.

14. The kit of claim 13, wherein the spacing of each area is 1 μm, 2 μm, 3 μm, 5 μm, 10 μm, 20 μm, 30 μm, 50 μm, 100 μm, 150 μm or in a range between any two of the values.

15. The kit of claim 12, wherein the spacing of one area is 2 μm, 3 μm, 5 μm, 10 μm, or in a range between any two of the values.

16. The kit of claim 12, wherein the difference between two spacing of each area is 0.5 μm, 1 μm, 2 μm, 3 μm, 5 μm, 10 μm, 20 μm, 30 μm, 50 μm, 100 μm, 150 μm, or in a range between any two of the values.

17. The kit of claim 1, wherein the signal measured in imaging area is cell or particle numbers, a colorimetric intensity, a transmitted light intensity, a fluorescence signal intensity, and/or an transmittance and/or absorptance.

18. The kit of claim 1, wherein the two plates are not aligned at their edges to create the flowing boundary of the sample by capillary force.

19. The kit of claim 1, wherein the sample collecting and transferring device and the sample distribution and imaging device each comprises an inner surface having a hydrophobic coating, hydrophilic coating, or both hydrophobic and hydrophilic coatings.

20. The kit of claim 1, wherein a coating is on at least an interior opposing surface of one or both of the plates.

21. The kit of claim 1, wherein the liquid sample for analyzing is pre-mixed with reagents before flowing into the device (a) or (b).

22. The kit of claim 1, wherein the reagent is coated with a slope distribution inside the device to balance the final concentration in the device (a) after adding the liquid sample.

23. The kit of claim 1, wherein there is a slow releasing coating with the reagent inside the device (a), the slow releasing layer delaying the reagents dissolving into the liquid sample after the sample become static after flowing in.

24. The kit of claim 1, wherein the slowing releasing time is 3 second, 5 second, 10 second or in a range between any of these values.

25. The kit of claim 1, wherein the slow releasing materials comprise albumin, carbomers, carboxymethyl cellulose, carrageenan, chitosan, dextrin, polyethylene glycol, polyvinylpyrrolidone, and/or polyvinyl alcohol.

26. The kit of claim 1, wherein an acridine orange is coated onto one or both of the two plates.

27. The kit of claim 1, wherein a Zwittergent is coated onto one or both of the two plates.

28. The kit of claim 1, wherein a Methylene blue and a Zwittergent are coated onto one or both of the two plates.

29. The kit of claim 1, wherein an acridine orange and a Zwittergent are coated onto one or both of the two plates.

30. The kit of claim 1, wherein a YOYO dye and a Zwittergent are coated onto one or both of the plates.

31. The kit of claim 1, wherein the device further comprises, on one or both plates, multi reagent layers including anti-conglutination, cell lysing, cell staining, release time control material layers, and their combinations.

32. The kit of claim 1, there is an array of spacers disposed between the two plates.

33. The kit of claim 32, wherein the spacers are attached on one of the plates or both plates, wherein the spacers can be used as physical support of the two plates, the geometry reference, dimensional reference, and/or optical reference in the measurements.

34. The kit of claim 32, wherein the lateral dimension of each spacer in each direction is the same or different.

35. The kit of claim 34, wherein the lateral dimension for each direction (x or y) is 1 nm or less, 3 nm or less, 5 nm or less, 7 nm or less, 10 nm or less, 20 nm or less, 30 nm or less, 40 nm or less, 50 nm or less, 100 nm or less, 200 nm or less, 500 nm or less, 800 nm or less, 1000 nm or less, 1 μm or less, 2 μm or less, 3 μm or less, 5 μm or less, 10 μm or less, 20 μm or less, 30 μm or less, 50 μm or less, 100 μm or less, 150 μm or less, 200 μm or less, 300 μm or less, or 500 μm or less, or in a range between any two of the values.

36. The kit of claim 32, wherein the period of spacer array is between 1 nm to 300 μm.

37. The kit of claim 32, wherein one dimension of each spacer is 0.1 mm, 0.2 mm, 0.5 mm, 1 mm, 2 mm, 5 mm, 10 mm, 20 mm or in a range between any of the two values.

38. The kit of claim 32, wherein each spacer is made of plastic, polymer, adhesive materials, dielectric materials.

39. The kit of claim 32, wherein the thickness variation of the spacers is less than 1%, less than 3%, less than 5%, less than 10% or in a range between any of the two values.

40. The kit of claim 32, wherein the spacers function as the adhesive structure to both plates.

41. The kit of claim 32, wherein there are separated bonding or adhesive structure between two chambers.

42. The kit of claim 41, wherein the bonding structure is a paste pad or paste lines.

43. The kit of claim 1, there are separated bonding or adhesive structure outside of two chambers.

44. A method of performing biological and chemical assays, comprising the steps:
(1) providing the kit of claim 1,
(2) collecting a sample into the sample collecting and transferring device, where the sample is mixed and stained by the reagent in the sample collecting and transferring device;
(3) transferring the sample from the sample collecting and transferring device by providing the external force to push out the sample to the sample landing Zone at the exterior location of the sample distribution and imaging device, where the sample flows into the cavity of the sample distribution and imaging device from the sample landing Zone and mix with reagent inside the sample distribution and imaging device, and distribute inside the sample distribution and imaging device; and
(4) imaging and analyzing the sample inside the sample distribution and imaging device.

45. The method of claim 44, wherein the liquid sample is whole blood, and an analyte to be analyzed is red blood cells, white blood cells and platelets.

46. The method of claim 45, wherein parameters measured in an imaging area is complete blood count, red blood cell count, hemoglobin, platelet count, white blood cell differentiation and count, and/or abnormal cell types.
